(19) United States
(12) Patent Application Publication
(10) Pub. No.: US 2022/0304985 A1
(43) Pub. Date: Sep. 29, 2022

(54) COMBINATION THERAPY FOR PROLIFERATIVE CONDITIONS

(71) Applicant: Avexxin AS, Trondheim (NO)

(72) Inventors: Berit Johansen, Trondheim (NO); Astrid Jullumstre Feuerherm, Trondheim (NO)

(21) Appl. No.: 17/611,053

(22) PCT Filed: May 15, 2020
(86) PCT No.: PCT/EP2020/063717

§ 371 Date: Nov. 12, 2021

(30) Foreign Application Priority Data
May 15, 2019 (GB) 1906864.2

(51) Int. Cl. A61K 31/426; A61K 31/5377; A61K 31/498; A61K 31/52; A61K 45/06

(52) U.S. Cl. CPC A61K 31/426; A61K 31/5377; A61K 31/498; A61K 31/52; A61K 45/06

(57) ABSTRACT

A pharmaceutical composition or combination product comprising certain thiazoles in combination with certain protein kinase inhibitors, in particular phosphatidylinositol-4,5-bisphosphate 3-kinase inhibitors (PI3K), such as buparlisib. The invention also relates to the use of said pharmaceutical composition or combination product for the treatment or prevention of proliferative conditions such as cancer, e.g. breast cancer or lymphoid cancer.

This invention relates to a pharmaceutical composition or combination product comprising certain thiazoles in combination with certain protein kinase inhibitors, in particular phosphatidylinositol-4,5-bisphosphate 3-kinase inhibitors (PI3K). The invention also relates to the use of said pharmaceutical composition or combination product for the treatment or prevention of proliferative conditions such as cancer, e.g. breast cancer or lymphoid cancer. The invention also relates to methods of treating or preventing proliferative conditions in patients comprising administration of the pharmaceutical composition or combination product of the invention to the patient.

### BACKGROUND

Basal-like breast cancer (BLBC), which represents ˜15% of all breast cancers, is an aggressive molecular subtype of the disease associated with poor prognosis. Most BLBCs are triple-negative (lacking expression of estrogen receptor, progesterone receptor, and human epidermal growth factor receptor 2) and thus unresponsive to currently available targeted therapies.

Leukaemias account for approximately 8% of all human malignancies. Human leukaemia can be divided into four subgroups based on the cell lineage and clinical manifestation. Acute leukaemias, acute myeloid leukaemia (AML) and acute lymphoid leukaemia (ALL), are associated with aggressive and usually fatal diseases, unless treated immediately. Both are most prevalent amongst young children. Due to their slow onset, chronic leukaemias, chronic myeloid leukaemia (CML) and chronic lymphoid leukaemia (CLL), are most commonly found in the older population.

Acute lymphoid leukaemia (ALL) represent 12% of all leukaemia cases, and approximately 80% of all leukaemia cases amongst children. The majority of ALL patients do not display an identifiable genetic or environmental cause. Genetic mutations, hereditary links and exposure to carcinogenic factors are however believed to be the most prominent risk factors. In ALL, early haemopoietic cells, called blast cells, accumulate in the bone marrow. This is also reflected in the clinical manifestation of ALL, characterised by anaemia and leucopenia as the bone marrow fails to exert its normal function.

Hence, new molecular targets for treatment of these cancers are called for.

The present inventors have devised a new combination therapy that targets proliferative conditions in general and breast cancer and lymphoid cancer in particular.

The invention relies on the combination of certain thiazoles compound and certain inhibitors of PI3K. The present inventors have surprisingly found that the combination of these two compounds leads to a combination therapy that works synergistically. In particular, the combination has been shown to synergistically reduce breast cancer and lymphoid cancer cell viability.

### SUMMARY OF INVENTION

Thus, viewed from one aspect the invention provides a pharmaceutical composition comprising:

(A) a compound of formula (I):

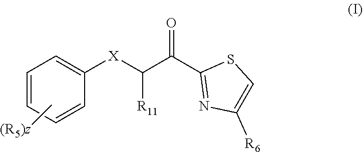

wherein X is O or S, preferably O;
R₆ is H, C₁₋₆alkyl, —(CH₂)ₚCOOH, —(CH₂)ₚCOOC₁₋₆alkyl, —(CH₂)ₚCONH₂, —(CH₂)ₚCONHC₁₋₆alkyl, and —(CH₂)ₚCON(C₁₋₆alkyl)₂;
R¹¹ is H or C₁₋₆ alkyl;
each R₅ is —OC₁₋₁₀alkyl, —SC₁₋₁₀alkyl, —C₁₋₁₂alkyl, or OAr²;
wherein Ar² is phenyl, optionally substituted with one or more halo;
each p is 0 to 3;
each z is 1 to 2;
or a pharmaceutically acceptable salt, or a hydrate or solvate; and
(B) a compound of formula (X)

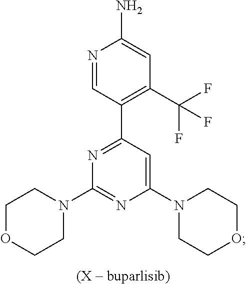 [SMILES: Nc1cc(C(F)(F)F)c(-c2cc(N3CCOCC3)nc(N3CCOCC3)n2)cn1]

or
a compound of formula (XI)

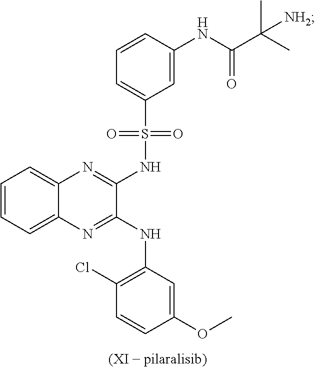 [SMILES: COc1ccc(Cl)c(Nc2nc3ccccc3nc2NS(=O)(=O)c2cccc(NC(=O)C(C)(C)N)c2)c1]

or a compound of general formula (XII)

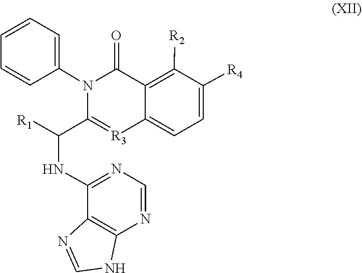

wherein R₁ is C₁₋₄ alkyl, such as Me or Et;
R₂ is H or Hal;
R₃ is N or CH; and
R₄ is H or Hal;
   or a pharmaceutically acceptable salt, or a hydrate or solvate thereof of any of formulae (X) to (XII).

Preferred compounds of formula (XII) are those of formula:

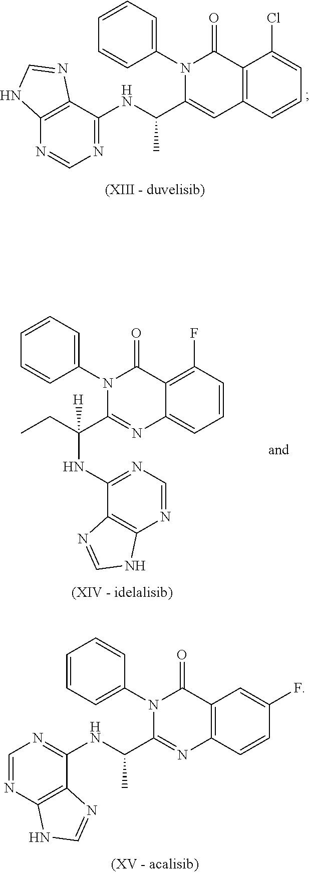 [SMILES: C[C@H](Nc1ncnc2[nH]cnc12)c1cc2cccc(Cl)c2c(=O)n1-c1ccccc1 ; CC[C@H](Nc1ncnc2[nH]cnc12)c1nc2cccc(F)c2c(=O)n1-c1ccccc1 ; C[C@H](Nc1ncnc2[nH]cnc12)c1nc2ccc(F)cc2c(=O)n1-c1ccccc1]

or a pharmaceutically acceptable salt, or a hydrate or solvate thereof.

Viewed from another aspect the invention provides a combination product for simultaneous, sequential or separate use comprising:
(A) a compound of formula (I):

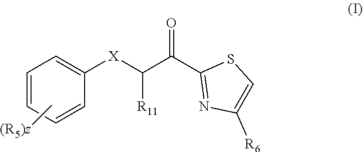

wherein X is O or S, preferably O
R₆ is H, C₁₋₆alkyl, —(CH₂)ₚCOOH, —(CH₂)ₚCOOC₁₋₆alkyl, —(CH₂)ₚCONH₂, —(CH₂)ₚCONHC₁₋₆alkyl, and —(CH₂)ₚCON(C₁₋₆alkyl)₂;
R¹¹ is H or C₁₋₆ alkyl;
each R₅ is —OC₁₋₁₀alkyl, —SC₁₋₁₀alkyl, —C₁₋₁₂alkyl, or OAr²;
wherein Ar² is phenyl, optionally substituted with one or more halo;
each p is 0 to 3;
each z is 1 to 2;
or a pharmaceutically acceptable salt, or a hydrate or solvate; and
(B) a compound of formula (X)

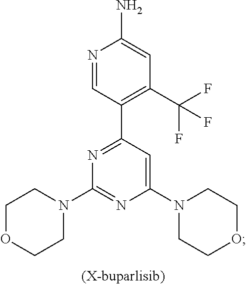 [SMILES: Nc1cc(C(F)(F)F)c(-c2cc(N3CCOCC3)nc(N3CCOCC3)n2)cn1]

or
a compound of formula (XI)

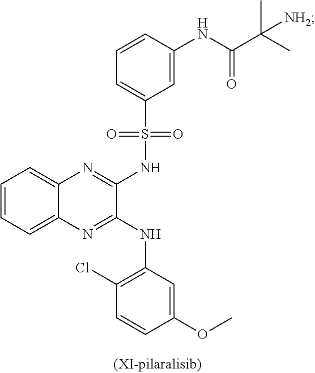 [SMILES: COc1ccc(Cl)c(Nc2nc3ccccc3nc2NS(=O)(=O)c2cccc(NC(=O)C(C)(C)N)c2)c1]

or a compound of general formula (XII)

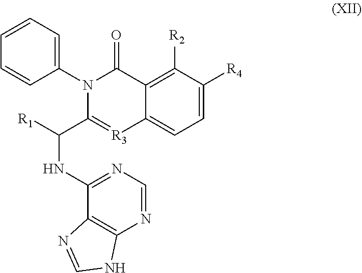

wherein R₁ is C₁₋₄ alkyl, such as Me or Et;
R₂ is H or Hal;
R₃ is N or CH; and
R₄ is H or Hal;
   or a pharmaceutically acceptable salt, or a hydrate or solvate thereof of any of formulae (X) to (XII).
   Viewed from another aspect the invention provides a pharmaceutical kit composition for simultaneous, sequential or separate use comprising a first composition comprising a compound (I) as herein defined and a pharmaceutically-acceptable diluent or carrier, and a second composition comprising a compound (X) to (XII) as herein defined and a pharmaceutically-acceptable diluent or carrier.

In particular, the invention relates to a pharmaceutical composition, combination product or kit as herein before defined in which the compound of formula (I) is:

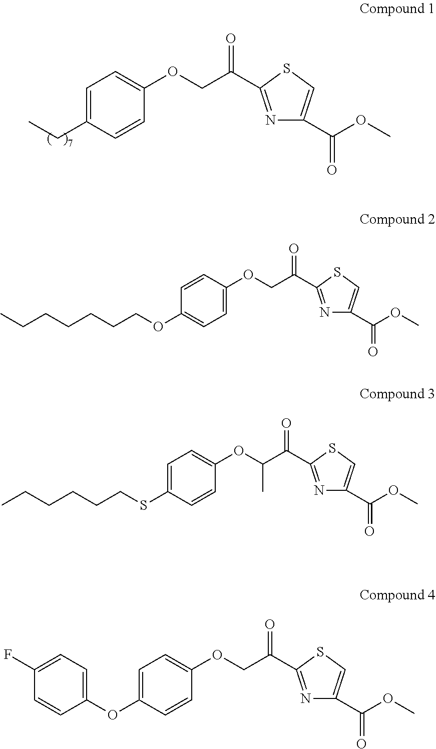

or a pharmaceutically acceptable salt, or a hydrate or solvate; and
a compound of formula (X) to (XII) as hereinbefore defined or a pharmaceutically acceptable salt, or a hydrate or solvate thereof.

Viewed from another aspect the invention provides a pharmaceutical composition or combination product as hereinbefore defined for use in the treatment or prevention of a proliferative disorder such as cancer, especially breast carcinoma or lymphoid cancer.

Viewed from another aspect the invention provides a method of treating or preventing a proliferative disorder such as cancer, especially breast carcinoma or lymphoid cancer in a patient in need thereof comprising administering to said patient, preferably a human, an effective amount of a pharmaceutical composition or combination product as herein before defined.

Viewed from another aspect the invention provides a method of treating, such as reducing symptoms of, or preventing a proliferative disorder such as cancer, especially breast carcinoma or lymphoid cancer in a patient in need thereof comprising administering to said patient, preferably a human, an effective amount of a compound of formula (I) and simultaneously, separately or sequentially administering to said patient a compound of formula (X) to (XII) as herein before defined. In sequential administration either compound can be administered first.

Viewed from another aspect the invention provides a method of treating, such as reducing symptoms of, or preventing a proliferative disorder such as cancer, especially breast carcinoma or lymphoid cancer, in a patient in need thereof comprising:
(i) identifying a patient who has received either a compound of formula (I) or a compound of formula (X) to (XII) as herein before defined respectively;
(ii) administering to said patient an effective amount of either a compound of formula (X) to (XII) or a compound of formula (I) as herein before defined so that said patient is administered with both a compound of formula (I) and a compound of formula (X) to (XII).

Viewed from another aspect the invention provides use of a composition or combination product as hereinbefore defined in the manufacture of a medicament for treating or preventing a proliferative disorder such as cancer, especially breast carcinoma or lymphoid cancer. Viewed from another aspect the invention provides a process for the preparation of a composition as hereinbefore defined comprising blending a compound of formula (I) and a compound of formula (X) to (XII) in the presence of at least one pharmaceutical excipient.

### DEFINITIONS

The term Hal means halide herein, such as Cl or F.

Where possible, the compounds of the invention, i.e. those of formula (I) to (V) or formula (X) to (XV) can be administered in salt, hydrate or solvate form, especially salt form or can be administered in non-salt form.

The invention relates both to a pharmaceutical composition in which compounds (I) and (X) to (XII) are blended together in a single composition and to a combination product such as a kit in which the active compounds are provided in separate compositions but are designed for administration simultaneously, separately or sequentially. Any method for treating or preventing a proliferative disorder as defined herein encompasses simultaneous, separate or sequential administration of the active components or administration of the composition of the invention.

A “combination” according to the invention refers to either a fixed combination in one dosage unit form, or a kit of parts for the combined administration where a compound of the formula (I) and its combination partner formula (X) to (XII) (also referred to as “combination partner” or “therapeutic agent”) may be administered independently at the same time or separately within time intervals, especially where these time intervals allow that the combination partners show a cooperative and preferably a synergistic effect.

A “combination product” as used herein means a product suitable for pharmaceutical use that results from the mixing or combining of more than one active ingredient and includes both fixed and non-fixed combinations of the active ingredients. The term “fixed combination” or “fixed dose” means that the active ingredients, e.g. a compound of formula (I) and its combination partner, a compound of formula (X) to (XII), are both administered to a patient simultaneously in the form of a single entity or dosage. The term “non-fixed combination” means that the active ingredients, e.g. a compound of formula (I) and the combination partner, a compound of formula (X) to (XII), are both administered to a patient as separate entities either simultaneously, concurrently or sequentially with no specific time limits, wherein such administration provides therapeutically effective levels of the two compounds in the body of the warm-blooded animal in need thereof.

All discussion below relating to preferred compounds of the invention is equally applicable to both these aspects of the invention.

### DETAILED DESCRIPTION OF THE INVENTION

This invention concerns a combination therapy of a compound of formula (I) and a compound of formula (X) to (XII). We have surprisingly found that this combination therapy results in synergy. Our results demonstrate a reduction in the viability of breast cancer cells or lymphoid cancer cells, the pharmaceutical composition or combination product offering a larger reduction than could have been expected from the use of individual compounds individually, i.e. the combination of the compounds produces an overall effect that is greater than the sum of the individual elements.

### Proliferative Disorder

This invention relates to a new combination therapy for proliferative disorders. Preferably, the composition of the invention is used for the treatment of a proliferative disease selected from a benign or malignant tumor, carcinoma of the brain, kidney, liver, adrenal gland, bladder, breast, lymphatic system, stomach, gastric tumors, ovaries, colon, rectum, prostate, pancreas, lung, vagina or thyroid, sarcoma, glioblastomas, multiple myeloma, leukemia or gastrointestinal cancer.

It is especially preferred if the proliferative disorder is a mammary carcinoma or lymphoid cancer, such as chronic lymphoid leukaemia or acute lymphoid leukaemia. The composition or combination product of the invention can target specifically metatstaic breast adenocarcinoma or acute lymphoid leukaemia.

### Compound A

The invention relies on the therapeutic combination of a compound of formula (I) and a compound of formula (X) to (XII). The compound of formula (I) is

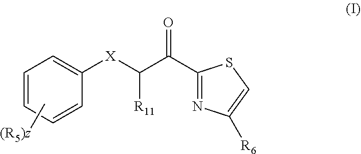

wherein X is O or S, preferably O;
R₆ is H, C₁₋₆alkyl, —(CH₂)ₚCOOH, —(CH₂)ₚCOOC₁₋₆alkyl, —(CH₂)ₚCONH₂, —(CH₂)ₚCONHC₁₋₆alkyl, —(CH₂)ₚCON(C₁₋₆alkyl)₂;
R¹¹ is H or C₁₋₆ alkyl;
each R₅ is —OC₁₋₁₀alkyl, —SC₁₋₁₀alkyl, —C₁₋₁₂alkyl, or OAr²;
wherein Ar² is phenyl, optionally substituted with one or more halo;
each p is 0 to 3;
each z is 1 to 2;
or a pharmaceutically acceptable salt, or a hydrate or solvate.

It is preferred if X is O.

It is preferred if R₆ is —COOC₁₋₆alkyl, or —CONHC₁₋₆alkyl, e.g. —COOC₁₋₁₂alkyl, or —CONHC₁₋₂alkyl.

It is preferred if R¹¹ is H or methyl, preferably H.

It is preferred if z is 1. It is preferred if p is 0.

It is preferred if the R₅ group is in the para position on the ring.

It is preferred if R₅ is —OC₄₋₁₀alkyl, —SC₄₋₁₀alkyl, —C₄₋₁₀alkyl, or OAr²;
wherein Ar² is phenyl, optionally substituted with one halo. Halo means halogen and is preferably Cl or F, especially F.

More preferably, the compound of formula (I) is of formula (II):

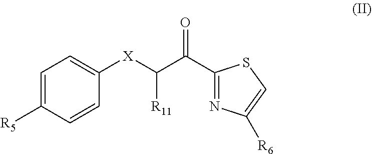

wherein X is O or S;
R₆ is H, C₁₋₆alkyl, —(CH₂)ₚCOOH, —(CH₂)ₚCOOC₁₋₆alkyl, —(CH₂)ₚCONH₂, —(CH₂)ₚCONHC₁₋₆alkyl, —(CH₂)ₚCON(C₁₋₆alkyl)₂,
R¹¹ is H or C₁₋₆ alkyl;
R₅ is —OC₁₋₁₀alkyl, —SC₁₋₁₀alkyl, —C₁₋₁₂alkyl, or OAr²;
Ar² is phenyl, optionally substituted with one or more halo;
each p is 0 to 3;
or a salt thereof.

More preferred compounds of formula (I) of the invention are those of formula (III):

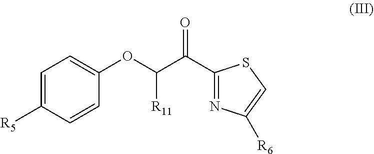

R₆ is —(C₁₋₁₂)ₚCOOC₁₋₆alkyl, or —(CH₂)ₚCONHC₁₋₆alkyl;
R¹¹ is H or methyl;
R₅ is —OC₁₋₁₀alkyl, —SC₁₋₁₀alkyl, —C₁₋₁₂alkyl, or OAr²;
Ar² is phenyl, optionally substituted with one halo;
each p is 0 to 3;
or a salt thereof.

More preferred compounds of formula (I) of the invention are those of formula (IV):

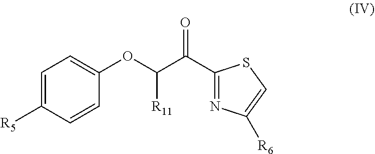

R₆ is —COOC₁₋₆alkyl, or —CONHC₁₋₆alkyl;
R¹¹ is H or methyl;
R₅ is —OC₁₋₁₀alkyl, —SC₁₋₁₀alkyl, —C₁₋₁₂alkyl, or OAr²;
Ar² is phenyl, optionally substituted with one halo;
or a salt thereof.

More preferred compounds of formula (I) of the invention are those of formula (V):

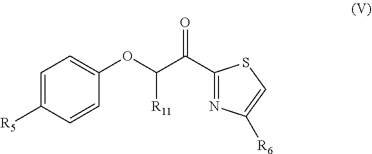

R₆ is —COOC₁₋₂alkyl;
R¹¹ is H or methyl;
R₅ is —OC₄₋₁₀alkyl, —SC₄₋₁₀alkyl, —C₄₋₁₂alkyl, or OAr₂;
Ar² is phenyl, optionally substituted with one halo;
or a salt thereof.

More preferred compounds of formula (I) of the invention are those of formula (VI):

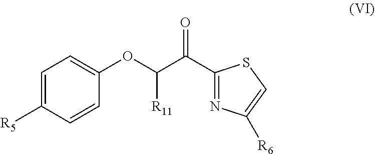

R₆ is —COOCH₃;
R¹¹ is H or methyl;
R₅ is —OC₅₋₈alkyl or —SC₅₋₈alkyl;
or a salt thereof.

Preferred compounds are:

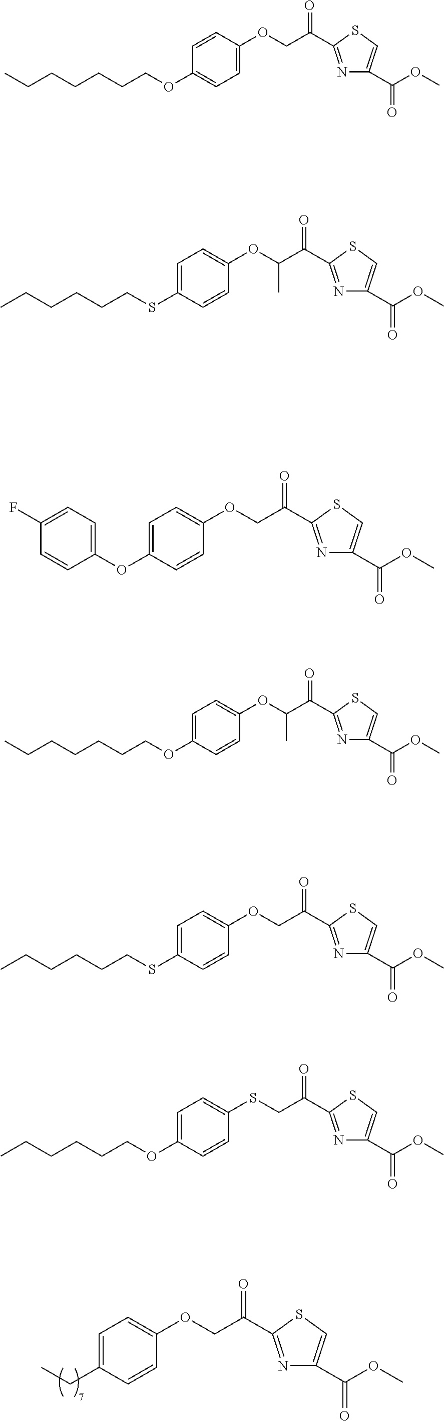

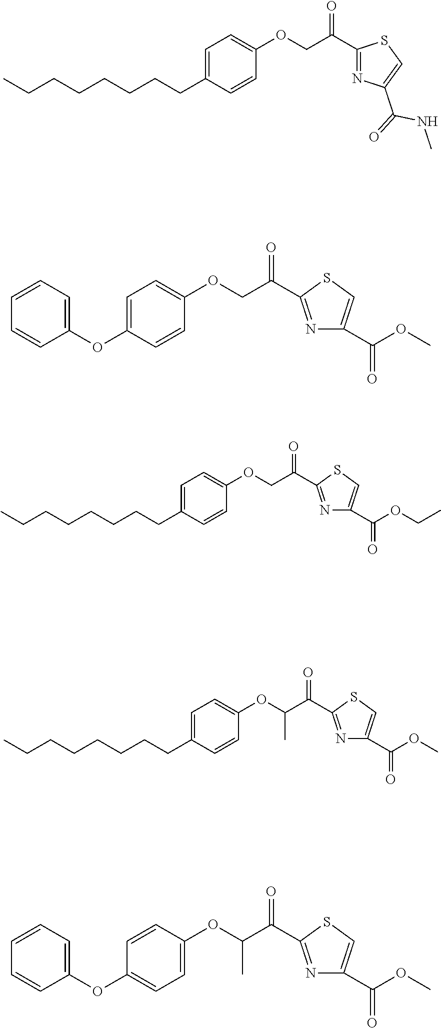

or salts thereof.

Especially preferred compounds are:

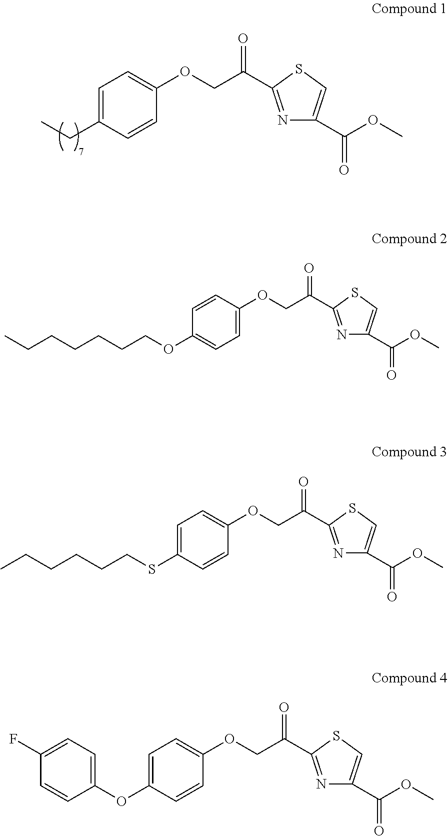

or a salt thereof.

### Compound B

The second component of the composition or combination product of the invention is a compound of formula (X) to (XII) as hereinbefore defined.

In compounds of general formula (XII):

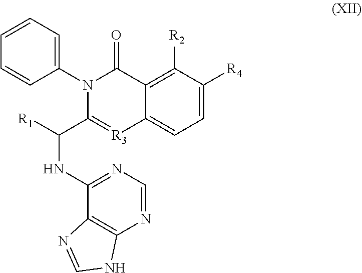

it is preferred if one of R₂ or R₄ is H and the other is Hal, e.g. F or Cl. It is preferred if R₄ is H and R₂ is Hal.

It is preferred if R₁ is methyl or ethyl.

In a preferred embodiment therefore, the invention provides a structure of formula (XII) in which:
R₁ is Me or Et;
R₂ is Hal;
R₃ is N or CH; and
R₄ is H.

Preferred options are:

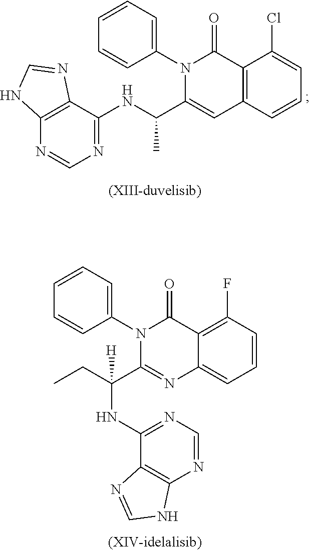 [SMILES: C[C@H](Nc1ncnc2[nH]cnc12)c1cc2cccc(Cl)c2c(=O)n1-c1ccccc1 ; CC[C@H](Nc1ncnc2[nH]cnc12)c1nc2cccc(F)c2c(=O)n1-c1ccccc1]

or acalisib (of formula XV).

In a further embodiment, pilaralisib may be used. In a most preferred embodiment, the compound is:

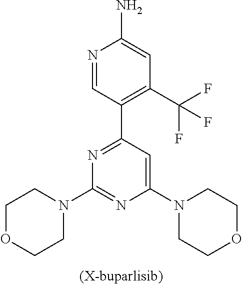 [SMILES: Nc1cc(C(F)(F)F)c(-c2cc(N3CCOCC3)nc(N3CCOCC3)n2)cn1]

or a salt thereof.

In a most preferred embodiment therefore the invention relates to a pharmaceutical composition or combination product comprising one or more of Compounds 1 to 4 defined above and buparlisib, especially compound 2 and buparlisib.

Where possible, the compounds of the invention, i.e. those of formula (I) to (V) or formula (X) to (XII) can be administered in salt, hydrate or solvate form, especially salt form.

Typically, a pharmaceutical acceptable salt may be readily prepared by using a desired acid. The salt may precipitate from solution and be collected by filtration or may be recovered by evaporation of the solvent. For example, an aqueous solution of an acid such as hydrochloric acid may be added to an aqueous suspension of a compound of formula (X) to (XII) and the resulting mixture evaporated to dryness (lyophilised) to obtain the acid addition salt as a solid. Alternatively, a compound of formula (X) to (XII) may be dissolved in a suitable solvent, for example an alcohol such as isopropanol, and the acid may be added in the same solvent or another suitable solvent. The resulting acid addition salt may then be precipitated directly, or by addition of a less polar solvent such as diisopropyl ether or hexane, and isolated by filtration.

Suitable addition salts are formed from inorganic or organic acids which form non-toxic salts and examples are hydrochloride, hydrobromide, hydroiodide, sulphate, bisulphate, nitrate, phosphate, hydrogen phosphate, acetate, trifluoroacetate, maleate, malate, fumarate, lactate, tartrate, citrate, formate, gluconate, succinate, pyruvate, oxalate, oxaloacetate, trifluoroacetate, saccharate, benzoate, alkyl or aryl sulphonates (e.g. methanesulphonate, ethanesulphonate, benzenesulphonate or p-toluenesulphonate) and isethionate. Representative examples include trifluoroacetate and formate salts, for example the bis or tris trifluoroacetate salts and the mono or diformate salts, in particular the tris or bis trifluoroacetate salt and the monoformate salt.

Compounds of formula (I) may be manufactured using known chemical synthetic routes. Synthesis methods are outlined in WO2014/118195 and WO2011/039563 as well as references cited therein.

Synthesis methods for the preparation of compounds of formula (X) to (XII) are described in the literature. These compounds are commercially available.

The weight ratio of the compounds of formula (I) to compounds of formula (X) to (XII) in composition or combination product of the invention will be guided by intended use, the age and general health of the subject, and other parameters known to those of skill. For example, a particular weight ratio suitable for certain applications may be 10 to 90 wt % to 90 to 10 wt %, such as 30 to 70 wt % to 70 to 30 wt %.

More preferably, the amounts of each compound are determined in molar terms, and the ratio of each is 5:1 to 1:5 moles, such as 2:1 to 1:2 moles. Often, the compounds are used in an equimolar amount for certain applications.

The amount of the compounds of the invention in the composition will often be determined by the physican depending on the dosage required.

The composition or combination product of the invention is proposed primarily for use in the treatment or prevention of proliferative disorders such as cancer.

By treating or treatment is meant at least one of:
(i). inhibiting the disease i.e. arresting, reducing or delaying the development of the disease or a relapse thereof or at least one clinical or subclinical symptom thereof, or
(ii). relieving or attenuating one or more of the clinical or subclinical symptoms of the disease.

By prevention is meant (i) preventing or delaying the appearance of clinical symptoms of the disease developing in a mammal.

The benefit to a subject to be treated is either statistically significant or at least perceptible to the patient or to the physician. In general a skilled man can appreciate when “treatment” occurs. It is particularly preferred if the composition or combination product of the invention are used therapeutically, i.e. to treat a condition which has manifested rather than prophylactically. It may be that the composition or combination product of the invention is more effective when used therapeutically than prophylactically.

The composition or combination product of the invention can be used on any animal subject, in particular a mammal and more particularly to a human or an animal serving as a model for a disease (e.g., mouse, monkey, etc.).

In order to treat a disease an effective amount of the active composition or combination product needs to be administered to a patient. A “therapeutically effective amount” means the amount of a composition or combination product that, when administered to an animal for treating a state, disorder or condition, is sufficient to effect such treatment. The “therapeutically effective amount” will vary depending on the composition or combination product, the disease and its severity and the age, weight, physical condition and responsiveness of the subject to be treated and will be ultimately at the discretion of the attendant doctor.

It may be that to treat cancer according to the invention that the composition or combination product of the invention has to be readministered at certain intervals. Suitable dosage regimes can be prescribed by a physician.

The composition or combination product of the invention typically comprises the active components in admixture with at least one pharmaceutically acceptable carrier selected with regard to the intended route of administration and standard pharmaceutical practice.

The term “carrier” refers to a diluent, excipient, and/or vehicle with which an active compound is administered. The pharmaceutical compositions of the invention may contain combinations of more than one carrier. Such pharmaceutical carriers are well known in the art. The pharmaceutical compositions may also comprise any suitable binder(s), lubricant(s), suspending agent(s), coating agent(s), and/or solubilizing agent(s) and so on. The compositions can also contain other active components, e.g. other drugs for the treatment of cancer.

It will be appreciated that pharmaceutical composition or combination products for use in accordance with the present invention may be in the form of oral, parenteral, transdermal, sublingual, topical, implant, nasal, or enterally administered (or other mucosally administered) suspensions, capsules or tablets, which may be formulated in conventional manner using one or more pharmaceutically acceptable carriers or excipients. The compositions of the invention could also be formulated as nanoparticle formulations.

However, for the treatment of cancer, the composition or combination product of the invention will preferably be administered orally or by parenteral or intravenous administration, such as injection. The composition or combination product may therefore be provided in the form of an tablet or solution for injection.

The pharmaceutical composition or combination product of the invention may contain from 0.01 to 99% weight—per volume of the active material. The therapeutic doses will generally be between about 10 and 2000 mg/day and preferably between about 30 and 1500 mg/day. Other ranges may be used, including, for example, 50-500 mg/day, 50-300 mg/day, 100-200 mg/day.

Administration may be once a day, twice a day, or more often, and may be decreased during a maintenance phase of the disease or disorder, e.g. once every second or third day instead of every day or twice a day. The dose and the administration frequency will depend on the clinical signs, which confirm maintenance of the remission phase, with the reduction or absence of at least one or more preferably more than one clinical signs of the acute phase known to the person skilled in the art.

It is within the scope of the present invention to administer the combination products described herein to a subject that has been exposed to, is being exposed to, or will be exposed to one or more anti-proliferative compounds and particularly those known to be used in many anti-cancer therapies. Non-limiting examples include aromatase inhibitors, anti-estrogens, topoisomerase I or II inhibitors microtubule active compounds, alkylating compounds, histone deacetylase inhibitors, and cyclooxygenase inhibitors such as those disclosed in W02006/122806 and references cited therein Choice of whether to combine a combination product of the invention with one or more of the aforementioned anti-cancer therapies will be guided by recognized parameters known to those of skill in the field, including the particular type of cancer being treated, the age and health of the subject, etc.

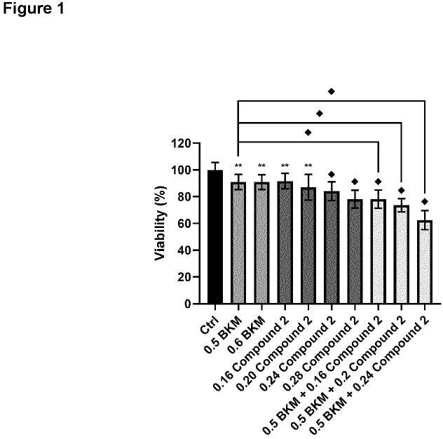

The invention is described further below with reference to the following non-limiting examples and figure. FIG. 1 shows combination treatment of BKM120 (μM) and Compound 2 (μM). The bars represent the mean relative viability±SD of three biological replicates. In FIG. 1, the 0.5 μm of BKM120 (buparlisib) used in combination corresponds to an expected inhibition of 10% Ctrl=control treatment, set to 100% viability. *=p<0.05, **=p<0.01, ***=p<0.001, ♦=p<0.0001.

### EXAMPLES

The following compounds were used in the examples:

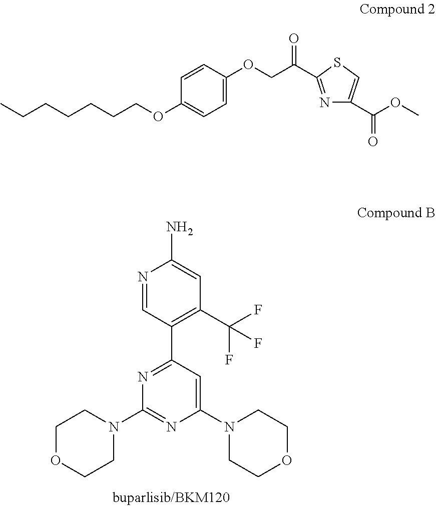 [SMILES: CCCCCCCOc1ccc(OCC(=O)c2nc(C(=O)OC)cs2)cc1 ; Nc1cc(C(F)(F)F)c(-c2cc(N3CCOCC3)nc(N3CCOCC3)n2)cn1]

### Methods

### Cell Culture.

The MDA-MB-436 cell line was from ATCC. The MDA-MB-436 cell line was established from a pleural effusion of a patient with metastatic breast adenocarcinoma. The cell line is of triple negative breast cancer, however the MDA-MB-436 cell line is classified as mesenchymal stem-like with high enrichment of genes for biological processes such as cell motility (Rho pathway), cellular differentiation and growth pathways (ALK pathway, TGF-β signalling and Wnt/β-catenin pathway).

MDA-MB-436 cells were maintained in RPMI-1640 culturing media supplemented with 10% FBS, 0.3 mg/mL L-glutamine and 0.1 mg/mL gentamicin, 10 mcg/mL insulin and incubated in 37° C. humidified atmosphere. Sub-culture using trypsin-EDTA 0.5% every 3-4 days with cell density of 2-3 million cells in a T75 cm2 culturing flask to ensure actively proliferating cells.

The CCRF-CEM (ATCC CCL-119) cell line is derived from T lymphoblasts from peripheral blood of a child with acute leukaemia. The cells were grown according to the recommendations of the supplier. The culture medium, RPMI-1640, was supplemented with 10 μL/mL glutamine, 2 μL/mL gentamicin and 10% foetal bovine serum (FBS) to make complete growth medium (CGM). The cells were incubated at 37oC in a humidified atmosphere with 5% CO2. Passaging was conducted every 2 - 4 days to maintain a cell density below 2.5×10⁶ cells/mL, and thus ensure a continuous proliferation. Cells were allowed to acclimatise for two weeks after establishment before they were utilised for experimental purposes and discarded after three months.

### Resazurin Viability Assay.

The MDA-MB-436 cells was seeded in 1×10⁴ cells/well. After 24 h of cultivation, when the cells were ˜30-50% confluent, the medium was replaced with serum free growth medium without gentamycin to ensure synchronization of the cells and to increase cell sensitivity to treatment. The MDA-MB-436 cells were starved in starvation media with insulin. The medium was replaced with fresh serum free medium with or without compound 2, NVP-BKM120 (Cayman Chemicals, US). The cells were observed under the microscope to evaluate possible morphology changes and signs of stress before the addition of resazurin according to the manufacturer's instructions (RnD Systems, UK). MDA-MB-436 cells were incubated for 72 h.

The CCRF-CEM cells were seeded at 4.0×10⁵ cells/mL 72 hours prior to the experiments to ensure that the cells were actively proliferating. Cells were seeded in 96 well plates at 5×10⁴ cells/well in 80 μL in either 10% FBS or in 0.5% FBS. The outermost wells were filled with medium instead of cells to avoid possible edge effects. The cells were incubated for 1 hour to allow them to settle prior to treatment with inhibitors. Working stocks of the inhibitors were made by diluting the inhibitors in dimethyl sulfoxide (DMSO). The final concentration of inhibitors was made by addition of medium. The concentration of DMSO in the treatments and control was standardised to 0.2%. The cells were treated with inhibitors for 24, 28, or 72 hours before adding resazurin.

Resazurin was metabolized for 1-2 h (37° C., 5% CO2) before fluorescence was read at 544 nm excitation and 590 nm emission wavelength (BioTek Synergy HT). The experiments were performed in series of eight wells per treatment and repeated 2-3 times.

### Combination Assay

To determine synergy, we assessed if the effect of the combination therapy was significantly greater than the effect by each monotherapy. Statistical significance between treatments were determined using Welch's ANOVA with Tamhane's T2 correction for multiple comparisons, p<0.05 was considered statistically significant.

For the CCRF-CEM experiments, Bliss Independence was also included to calculate a combination index (CI) for the investigated combinations. The combination index was calculated using:

$$CI = \frac{{E}_{A} + {E}_{B} - ( {E}_{A} \times {E}_{B} )}{{E}_{AB}}$$

where CI is the combination index, E_{A} represent the effect of drug A given as a monotherapy, E_{B} represent the effect of drug B given as a monotherapy and E_{AB} represent drugs A and B given in combination at the same concentrations as the monotherapies. A CI of less than 1 indicates synergy and a CI of more than 1 indicate antagonism, whereas a CI of 1 is indicative of additivism.

### Example 1

Dose-dependent Inhibition is Obtained for PI3K Inhibitor and cPLA2α Inhibitor Compound 2

It has been demonstrated that PI3K and cPLA2α inhibitors both effectively reduce cell proliferation in BLBC/TNBC. Dose-responses of isoform specific PI3K inhibitors and selective cPLA2α inhibitors in the MDA-MB-436 cells were performed prior to co-treatment experiments with PI3K inhibitor and compound 2.

Compound 2 demonstrated a dose-response relationship within the range of 27 μM and the IC50 value was 10.4 μM (Table 1). The viability was reduced significantly, for cells treated with 9 μM and above, compared to the untreated control set to 100% viability. The viability decreased abruptly and the standard deviation was high around the IC50 value. The slope of BKM120 decreased only modestly within the equivalent range. The PI3K inhibitor was more potent than compound 2 with IC50 of 1.2 μM (Table 1).

TABLE 1
Calculated IC50-values of pan PI3K inhibitor BKM120 and
the cPLA2α inhibitor Compound 2 on BLBC/TNBC cell
line MDA-MB-436. (72 h treatment, n = 3)

| | | IC50 |
|---|---|---|
| | | 72 h |
| Inhibitor | Target | (μM) |
| Buparlisib/BKM120 | Pan PI3K | 1.2 |
| | p110α/β/δ/γ | |
| Compound 2 | cPLA2α | 10.4 |

### Example 2

Based on the initial experiments, sub-optimal doses of cPLA2α inhibitors and PI3K inhibitor in the co-treatment experiments were identified. Sub-optimal doses of compound 2 in the MDA-MB-436 cell line were 5.0 μM, 6.5 μM, 7.5 μM, and 0.4 μM for BKM120.

The effects of proliferation in combination experiment for the MDA-MB-436 cells were studied at 72 h post-treatment. The combination of compound 2 with BKM120 showed synergism in all the doses tested and approximately two fold change of inhibition was observed when the two inhibitors were combined (Table 2). An additional of 25±5% inhibition was observed when 5 μM compound 2 was combined with 0.4 μM BKM120. In addition, the higher dose of compound 2 (6.5 μM) in combination with 0.4 μM BKM120 showed 42±10% additional effects and 45±2% in the highest dose of compound 2 (7.5 μM) in the combination experiment.

TABLE 2
Viability of MDA-MB-436 cells after treatment with sub-optimal doses of
the pan-PI3K inhibitor BKM120 and the cPLA2α inhibitor compound 2 alone
and in combination after 72 h. The viability was measured in percentage
relative to the untreated control set to 100%. P-value ****<0.0001

| | | % Viability |
|---|---|---|
| Treatment | Target | 72 h |
| Cmpd 2 (5 μM) | cPLA2α | 92 ± 3 |
| Cmpd 2 ( 6.5 μM) | cPLA2α | 82 ± 6 |
| Cmpd 2 (7.5 μM) | cPLA2α | 76 ± 4 |
| Cmpd 2 (10 μM) | cPLA2α | 60 ± 10 |
| BKM120 (1.1 μM) | Pan-PI3K | 45 ± 10 |
| | p110α/β/δ/γ | |
| BKM120 (0.4 μM) | Pan-PI3K | 84 ± 7 |
| | p110α/β/δ/γ | |
| Cmpd 2 (5 μM) + | cPLA2α + Pan-PI3K | 51 ± 5 *** |
| BKM120 (0.4 μM) | p110α/β/δ/γ | |
| Cmpd 2 (6.5 μM ) + | cPLA2α + Pan-PI3K | 24 ± 10 *** |
| BKM120 (0.4 μM) | p110α/β/δ/γ | |
| Cmpd 2 (7.5 μM ) + | cPLA2α + Pan-PI3K | 15 + 2 *** |
| BKM120 (0.4 μM) | p110α/β/δ/γ | |

### Discussion

Co-treatment of PI3K inhibitors such as BKM120 (pan-PI3K inhibitor), with the cPLA2α inhibitor compound 2 acts synergistically on reducing cell viability in the MDA-MB-436 cells. The observations that synergistic effects on cancer cell viability are obtained when compound 2 is combined with either of the PI3K inhibitor BKM120 indicate that the co-treatment strategy may be an effective treatment strategy in reducing cell viability and reducing resistance in highly aggressive breast cancer such as BLBC/TNBC.

### Example 3

Compound 2 and BKM120 were used in a CCRF-CEM cell line. Based on the established dose and time dependency for Compound 2 and BKM120, a number of combination experiments were performed in medium with the ideal serum concentration. Suboptimal doses of each compound, typically corresponding to 10-20% inhibition of viability were used to investigate possible synergy.

In Table 3, inhibition is presented as reduction in viability determined by the resazurin assay, with corresponding combination index (CI) for selected combinations of low dose BKM120 with Compound 2.

When assessing the CIs in Table 3, all the tested combinations of low dose BKM120 and Compound 2 indicate synergy as the CI values are all less than 1. As seen in FIG. 1, the reduction in viability for this combination is statistically significant from 0.6 μM BKM120 alone. Furthermore, all combination treatments are both statistically significant from the control treatment (p<0.001), as well as from the low dose of BKM120 given as a monotherapy.

TABLE 3
The total inhibition for the combinations between BKM120
and Compound 2 presented as the mean ± SD
with corresponding combination index (CI).

| BKM120 (μM) | Compound 2 (μM) | Total inhibition (%) | CI |
|---|---|---|---|
| 0.5 | 0.16 | 21.8 ± 6.8 | 0.74 |
| 0.5 | 0.20 | 26.4 ± 4.9 | 0.80 |
| 0.5 | 0.24 | 37.5 ± 7.2 | 0.62 |
| 0.6 | 0.12 | 21.7 ± 11.1 | 0.78 |
| 0.6 | 0.16 | 22.7 ± 13.1 | 0.75 |
| 0.6 | 0.20 | 30.3 ± 9.4 | 0.66 |

1. A combination product comprising:
(A) a compound of formula (I): 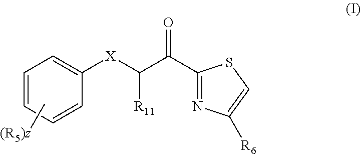
wherein X is O or S, preferably O;
R₆ is H, C₁₋₆alkyl, —(CH₂)ₚCOOH, —(CH₂)ₚCOOC₁₋₆alkyl, —(CH₂)ₚCONH₂, —(CH₂)ₚCONHC₁₋₆alkyl, and —(CH₂)ₚCON(C₁₋₆alkyl)₂;
R¹¹ is H or C₁₋₆ alkyl;
each R₅ is —OC₁₋₁₀alkyl, —SC₁₋₁₀alkyl, —C₁₋₁₂alkyl, or OAr²;
wherein Ar² is phenyl, optionally substituted with one or more halo;
each p is 0 to 3;
each z is 1 to 2;
or a pharmaceutically acceptable salt, or a hydrate or solvate thereof; and
(B) a compound of formula (X) 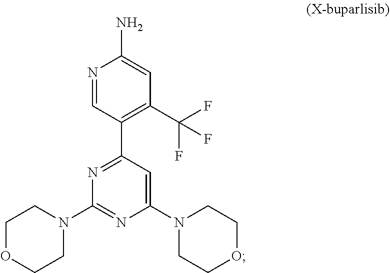 [SMILES: Nc1cc(C(F)(F)F)c(-c2cc(N3CCOCC3)nc(N3CCOCC3)n2)cn1]
or
a compound of formula (XI) 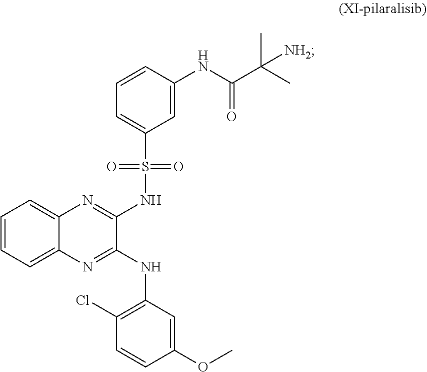 [SMILES: COc1ccc(Cl)c(Nc2nc3ccccc3nc2NS(=O)(=O)c2cccc(NC(=O)C(C)(C)N)c2)c1]
or a compound of general formula (XII) 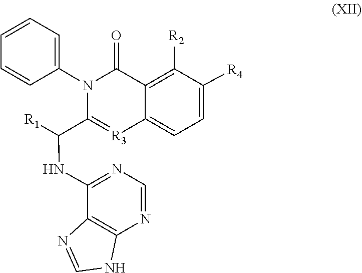
wherein R₁ is C₁₋₄ alkyl, such as Me or Et;
R₂ is H or Hal;
R₃ is N or CH; and
R₄ is H or Hal;
or a pharmaceutically acceptable salt, or a hydrate or solvate thereof of any of formulae (X) to (XII).

2. A pharmaceutical composition comprising:
(A) a compound of formula (I): 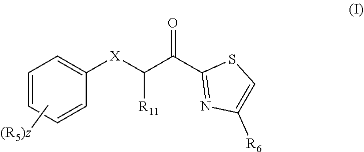
wherein X is O or S, preferably O;
R₆ is H, C₁₋₆alkyl, —(CH₂)ₚCOOH, —(CH₂)ₚCOOC₁₋₆alkyl, —(CH₂)ₚCONH₂, —(CH₂)ₚCONHC₁₋₆alkyl, and —(CH₂)ₚCON(C₁₋₆alkyl)₂;
R¹¹ is H or C₁₋₆ alkyl;
each R5 is —OC₁₋₁₀alkyl, —SC₁₋₁₀alkyl, —C₁₋₁₂alkyl, or OAr²;
wherein Ar² is phenyl, optionally substituted with one or more halo;
each p is 0 to 3;
each z is 1 to 2;
or a pharmaceutically acceptable salt, or a hydrate or solvate thereof; and
(B) a compound of formula (X) 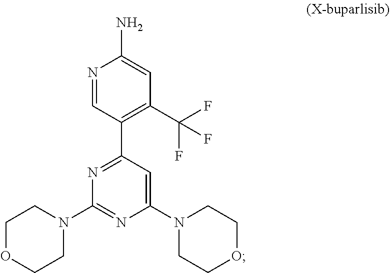 [SMILES: Nc1cc(C(F)(F)F)c(-c2cc(N3CCOCC3)nc(N3CCOCC3)n2)cn1]
or
a compound of formula (XI) 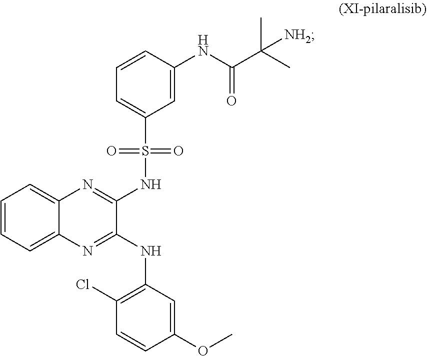 [SMILES: COc1ccc(Cl)c(Nc2nc3ccccc3nc2NS(=O)(=O)c2cccc(NC(=O)C(C)(C)N)c2)c1]
or a compound of general formula (XII) 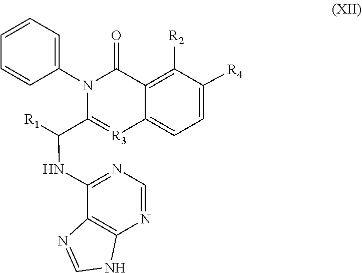
wherein R₁ is C₁₋₄ alkyl, such as Me or Et;
R₂ is H or Hal;
R₃ is N or CH; and
R₄ is H or Hal;
or a pharmaceutically acceptable salt, or a hydrate or solvate thereof of any of formulae (X) to (XII).

3. A pharmaceutical composition for simultaneous, sequential or separate use comprising a kit comprising a first composition comprising a compound (I) as defined in claim 1 and a pharmaceutically-acceptable diluent or carrier, and a second composition comprising a compound (X) to (XII) as defined in claim 1 and a pharmaceutically-acceptable diluent or carrier.

4. A composition or product as claimed in any preceding claim wherein said compound of formula (I) is: 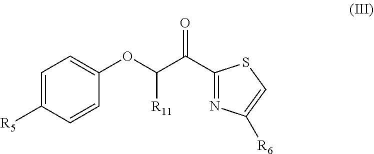
R₆ is —(CH₂)ₚCOOC₁₋₆alkyl, or —(CH₂)ₚCONHC₁₋₆alkyl;
R¹¹ is H or methyl;
R₅ is —OC₁₋₁₀alkyl, —SC₁₋₁₀alkyl, —C₁₋₁₂alkyl, or OAr²;
Ar² is phenyl, optionally substituted with one halo;
each p is 0 to 3;
or a salt thereof.

5. A composition or product as claimed in any preceding claim wherein said compound of formula (I) is: 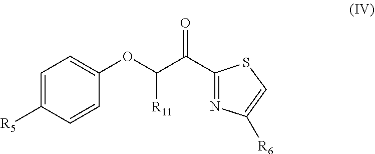
R₆ is —COOC₁₋₆alkyl, or —CONHC₁₋₆alkyl;
R¹¹ is H or methyl;
R₅ is —OC₁₋₁₀alkyl, —SC₁₋₁₀alkyl, —C₁₋₁₂alkyl, or OAr²;
Ar² is phenyl, optionally substituted with one halo;
or a salt thereof.

6. A composition or product as claimed in any preceding claim wherein said compound of formula (I) has the formula: 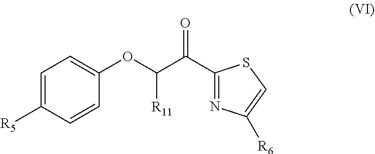
R₆ is —COOCH₃;
R¹¹ is H or methyl;
R₅ is —OC₅₋₈alkyl or —SC₅₋₈alkyl;
or a salt thereof.

7. A composition or product as claimed in any preceding claim where the compound of formula (I) is 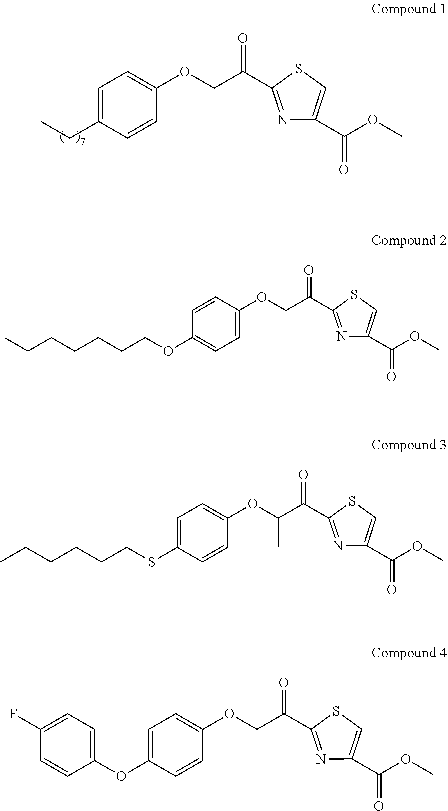
or a salt thereof.

8. A composition or product as claimed in any preceding claim where the compound of formula (I) is 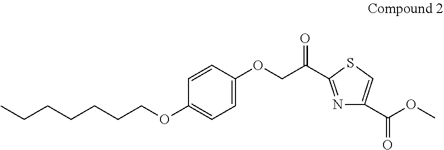 [SMILES: CCCCCCCOc1ccc(OCC(=O)c2nc(C(=O)OC)cs2)cc1]
or a salt thereof.

9. A composition or product as claimed in any preceding claim in which said compound B is of formula (X) 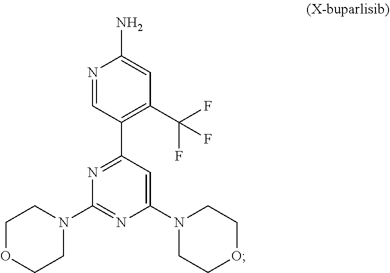 [SMILES: Nc1cc(C(F)(F)F)c(-c2cc(N3CCOCC3)nc(N3CCOCC3)n2)cn1]
or a compound of formula (XI) 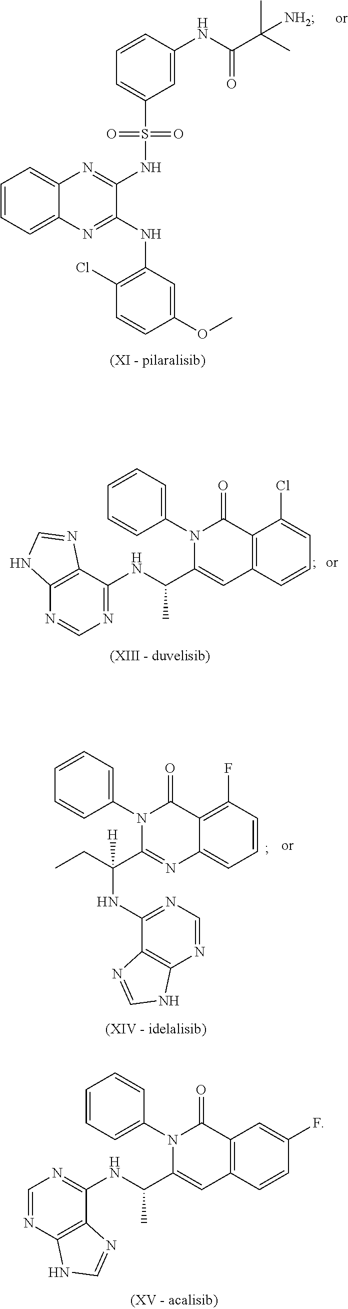 [SMILES: COc1ccc(Cl)c(Nc2nc3ccccc3nc2NS(=O)(=O)c2cccc(NC(=O)C(C)(C)N)c2)c1 ; C[C@H](Nc1ncnc2[nH]cnc12)c1cc2cccc(Cl)c2c(=O)n1-c1ccccc1 ; CC[C@H](Nc1ncnc2[nH]cnc12)c1nc2cccc(F)c2c(=O)n1-c1ccccc1 ; C[C@H](Nc1ncnc2[nH]cnc12)c1cc2ccc(F)cc2c(=O)n1-c1ccccc1]
or a salt thereof.

10. A composition or product as claimed in any preceding claim in which said compound B is buparlisib or a salt thereof.

11. A composition or product as claimed in any preceding claim in which said compound of formula (I) is 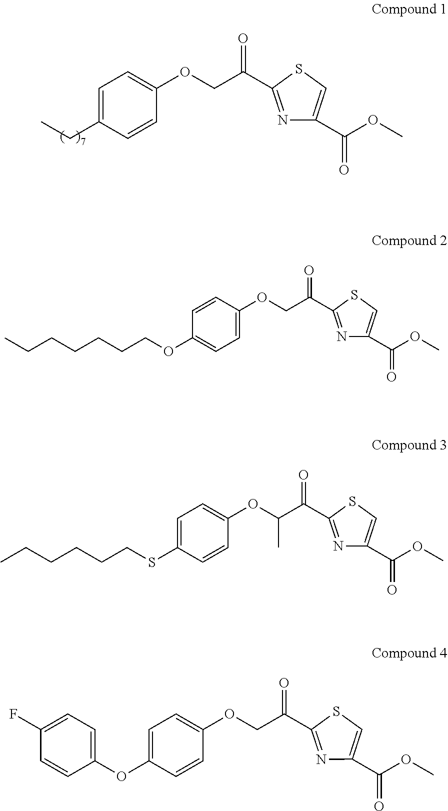
or a salt thereof; and the compound (B) is of formula (X) 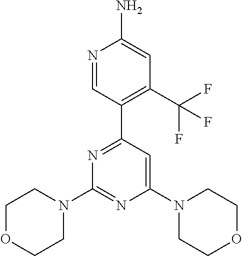 [SMILES: Nc1cc(C(F)(F)F)c(-c2cc(N3CCOCC3)nc(N3CCOCC3)n2)cn1]
or a salt thereof.

12. A pharmaceutical composition or combination product as claimed in claims 1 to 11 for use in the treatment or prevention of a proliferative disorder such as cancer, especially breast carcinoma or lymphoid cancer.

13. A method of treating, such as reducing symptoms of, or preventing a proliferative disorder such as cancer, especially breast carcinoma or lymphoid cancer in a patient in need thereof comprising administering to said patient, preferably a human, an effective amount of a composition or combination product as claimed in claims 1 to 11.

14. A method of treating, such as reducing symptoms of, or preventing a proliferative disorder such as cancer, especially breast carcinoma or lymphoid cancer in a patient in need thereof comprising administering to said patient, preferably a human, an effective amount of a compound of formula (I) as defined in claims 1 to 11 and simultaneously, separately or sequentially administering to said patient a compound of formula (X) to (XII) as defined in claims 1 to 11.

15. A method of treating such as, reducing symptoms of, or preventing a proliferative disorder such as cancer, especially breast carcinoma or lymphoid cancer, in a patient in need thereof comprising:
(i) identifying a patient who has received either a compound of formula (I) as defined in claims 1 to 11 or a compound of formula (X) to (XII) as defined in claims 1 to 11 respectively;
(ii) administering to said patient an effective amount of either a compound of formula (X) to (XII) or a compound of formula (I) as defined in claims 1 to 11 so that said patient is administered with both a compound of formula (I) and a compound of formula (X) to (XII).

16. Use of a composition or combination product as claimed in claims 1 to 11 in the manufacture of a medicament for treating or preventing a proliferative disorder such as cancer, especially breast carcinoma or lymphoid cancer.

17. The combination product of any of claims 1 to 11 wherein the product is a fixed combination or non-fixed combination.

18. The pharmaceutical composition or combination product of any of claims 1 to 11 in combination with one or more anti-proliferative compounds for use in cancer therapy.

19. The pharmaceutical composition or combination product for use as claimed in 18, wherein the anti-proliferative compound is selected from the group consisting of aromatase inhibitors, antiestrogens, topoisomerase I or II inhibitors, and microtubule active compounds, alkylating compounds, histone deacetylase inhibitors, and cyclooxygenase inhibitors.
